(19) United States
(12) Patent Application Publication
(10) Pub. No.: US 2011/0212461 A1
(43) Pub. Date: Sep. 1, 2011

(54) PREDICTION OF CARDIOTOXICITY

(76) Inventors: Hans Marcus Ludwig Bitter, Montclair, NJ (US); Kyle Louis Kolaja, Montclair, NJ (US); Preeti Dhawan, San Jose, CA (US); Hirdesh Uppal, San Ramon, CA (US); Nina Gonzaludo, Palo Alto, CA (US)

(21) Appl. No.: 12/713,976

(22) Filed: Feb. 26, 2010

(51) Int. Cl. G01N 33/53; C12M 1/40

(52) U.S. Cl. 435/7.1; 435/287.9

(57) ABSTRACT

The likelihood that a compound will exhibit cardiotoxicity in an in vitro or in vivo assay predicted by the ability of the compound to inhibit at least one kinase from a selected group.

### FIELD OF THE INVENTION

This invention relates generally to the field of toxicology. More particularly, the invention relates to methods for predicting cardiotoxicity, and methods for screening compounds for potential cardiotoxicity.

### BACKGROUND OF THE INVENTION

The heart is an adaptive organ for pumping blood, responding to changing needs by modifying contractile strength and beating rate. The cardiac myocyte is the principal cell in the heart; it coordinates contraction and has the capability to sense a large number of hormonal, neural, electrical and mechanical inputs through a variety of cell surface and nuclear receptors. Myocytes are also targets of an extraordinary number of physiological and pharmacological agents, because of the critical need to regulate contraction strength and heart rate, and their importance in several cardiovascular diseases.

Primary cells isolated from intact heart have been an important model for study because there are no cell lines that maintain the unique rod shaped morphology and complement of proteins necessary for cardiac function. In serum-free culture, adult cardiac myocytes from guinea pigs, rats, mouse and rabbits are usually quiescent and retain their viability and unique rod-shaped morphology for at least a few days. These cells maintain highly organized membrane and myofibrillar structures that support contractions induced by electrical or pharmacological stimulation, and are amenable to viral-mediated expression of exogenous proteins. But similarly successful culture of human cardiac myocytes has been more challenging and not possible, perhaps because of difficulties in enzymatic isolation of healthy myocytes and unique variables for relatively long-term culture. As a consequence, less is known about human cardiac myocyte physiology.

An understanding of cardiotoxicity and of the difficulties in predicting cardiotoxic potential requires insight into the molecular basis of the cardiac function. The understanding of molecular mechanisms of cardiotoxicity has shown that a multitude of extra cellular factors, intracellular factors, transcriptional events and signaling pathways are involved. Thus a large number of players have been shown to be key determinants in the orchestration of a multitude of these pathways to maintain normal cardiac function. Moreover, if dysregulated or inhibited, these extra cellular factors, intracellular factors, transcriptional events and signaling pathways cause the toxicities observed in adverse cardiovascular events. The development of targeted therapies, inhibitors, and drugs has shown some significant liabilities with regards to cardiotoxicity especially in the area of cancer therapy.

Recently, progress has been made in determining basic mechanisms underlying the cardiotoxicity of drugs. There are two key features to clarify for each drug, small molecule compound, ligand, or protein/biotherapuetic that show cardiotoxicity. First, determining the mechanisms of toxicity requires the identification of the specific target responsible for cardiotoxicity. The identification of targets mediating cardiotoxicity can also help to guide future drug development, because some of these molecules or proteins are likely to be ‘bystander’ targets that have no role in the disease indication that a given drug is being developed for and there is therefore no need for the drug to inhibit them. Second, there is a requirement for delineating the mechanisms of toxicity so that the signaling pathways that transduce the toxicity are identified. In some instances, the pathway that leads to cardiomyocyte dysfunction or death will not be the same as the pathway that is crucial for drug action. Therefore, strategies could be developed to block the drug-induced pathways that lead to toxicity but to leave the drug's therapeutic pathways intact.

The development of drugs that inhibit the activity of certain tyrosine kinases for cancer therapy have been associated with toxicity to the heart (Force et al., Drug Discovery Today (2008) 13(17/18), 778-784; Will et al., Toxicological Scineces (2008) 106(1), 153-161). The development of kinase inhibitors (KIs) creates many opportunities for toxicity, not only as a result of the inhibition of desired targets but, probably much more importantly, due to the inhibition of off-target kinases. Cardiotoxicity of a targeted agent was first reported for trastuzumab, the monoclonal antibody that targets the ERBB2 receptor and adverse cardiac effects have also been reported after treatment of patients with imatinib, and are mentioned in the prescribing information for dasatinib (Sprycel), sunitinib (Sutent), sorafenib (Nexavar) and bevacizumab (Avastin). Cardiotoxicity is not associated with all kinase inhibitors because it is not observed with certain other KIs, such as those that target the epidermal growth factor receptor. Therefore, cardiotoxicity needs to be determined for each agent on a case-by-case basis.

### SUMMARY OF THE INVENTION

We have now invented a method for predicting which compounds will demonstrate positive (i.e., cardiotoxicity) results in in vivo toxicity studies, using a method that is faster, uses smaller quantities of reagents, is easily automated, and is much cheaper than cardiotoxicity testing in vivo.

One aspect of the invention is a method for predicting the cardiotoxicity of a compound, the method comprising providing a test compound; and determining the ability of the compound to inhibit the kinase activity of a number of kinases selected from the group consisting of CSF1R, KIT, FYN, PDGFR beta, FGR, LCK, Ephrin Receptor B1, FRK, ABL1, PDGFR alpha, and HCK.

Another aspect of the invention is the method for screening candidate compounds for potential cardiotoxicity, comprising providing a plurality of compounds; and determining the ability of each compound to inhibit the kinase activity of a number of kinases selected from the group consisting of CSF1R, KIT, FYN, PDGFR beta, FGR, LCK, Ephrin Receptor B1, FRK, ABL1, PDGFR alpha, and HCK.

### DETAILED DESCRIPTION OF THE INVENTION

All publications cited in this disclosure are incorporated herein by reference in their entirety.

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

The term “cardiotoxicity” as used herein refers to compounds that cause direct or indirect injury to cardiomyocytes and the myocardium that may manifest in certain clinical symptoms which may include: congestive heart failure, ischemia, hypotension, hypertension, arrhythmias (e.g. bradycardia), edema, QT prolongation and conduction disorders, and thromboembolism.

The term “test compound” refers to a substance which is to be tested for cardiotoxicity. The test compound can be a candidate drug or lead compound, a chemical intermediate, environmental pollutant, a mixture of compounds, and the like.

The term “kinase” refers to an enzyme capable of attaching and/or removing a phosphate group from a protein or molecule. “Inhibition of kinase activity” refers to the ability of a compound to reduce or interfere with such phosphatase activity. As binding affinity of a small molecule for a given kinase correlates well with the ability of said molecule to inhibit the kinase activity, binding affinity is considered synonymous with kinase activity herein, and high binding affinity is considered equivalent to high kinase inhibitory activity.

The term “model kinase” refers to the following set of kinases (also identified by accession numbers in parentheses): CSF1R (NP_{—}005202.2), KIT (NP_{—}000213.1), FYN (NP_{—}694592.1), PDGFR beta (NP_{—}002600.1), FGR (NP_{—}005239.1), LCK (NP_{—}005347.3), Ephrin Receptor B1 (NP_{—}004432.1), FRK (NP_{—}002022.1), ABL1 (NP_{—}005148.2), PDGFR alpha (NP_{—}006197.1), and HCK (NP_{—}002101.2).

The term “potential model kinase” refers to the following set of kinases (also identified by accession numbers in parentheses): ABL2 (NP_{—}005149.3), LYN (NP_{—}002341.1), ZAK (NP_{—}598407.1), Ephrin Receptor B2 (NP_{—}059145.2), YES1 (NP_{—}005424.1), MAP4K4 (NP_{—}663719.1), PKN1 (NP_{—}998725.1), BRAF (NP_{—}004324.2), DDR2 (CAA52777.1), MAP4K5 (NP_{—}006566.2), and STK24 (NP_{—}003567.2).

All patents and publications identified herein are incorporated herein by reference in their entirety.

### General Method

The invention provides a method for quickly determining the likelihood that a given compound will exhibit cardiotoxicity in an in vivo or in vitro toxicity assay by examining the interaction between the compound and a number of kinases (kinase binding and/or inhibition). As kinase inhibition and/or binding can be determined quickly, and by using automated methods, the method of the invention enables high-throughput screening of compounds for cardiotoxicity (or lack thereof).

In practice, binding and inhibition can be determined using methods known in the art. See, for example, M. A. Fabian et al., Nature Biotechnol (2005) 23:329-36, incorporated herein by reference in full. In general, the binding affinity of a compound for a given kinase correlates well with the ability of the compound to inhibit the activity of that kinase, so that binding affinity is a reliable substitute for inhibitory activity. Binding affinity may be determined by a variety of methods known in the art; for example by competitive assay using an immobilized kinase (or an immobilized test compound, or an immobilized competing ligand, any of which may be labeled). Compounds and kinases can be immobilized by standard methods, for example by biotinylation and capture on a streptavidin-coated substrate.

Thus, one can prepare a test substrate having, for example, a plurality of immobilized kinases, preferably comprising the set of model kinases identified herein:

The kinases can be immobilized directly (i.e., by adsorption, covalent bond, or biotin-avidin binding or the like) to the surface, or indirectly (for example by binding to a ligand that is tethered to the surface by adsorption, covalent bond, biotin-avidin or other linkage). The kinases are then contacted with the test compound(s), and the affinity (or enzyme inhibition) determined, for example by measuring the binding of labeled compound or loss of labeled competitor.

The kinase affinity of each compound is measured against at the kinases comprising the model kinase group. A compound with high total activity (for example, demonstrating high affinity for five of the eleven kinases) has a high likelihood of cardiotoxicity: this compound is predicted to test positive for cardiotoxicity in either an in vitro or in vivo test system. A compound having low total activity (for example, showing only low affinity for the kinases in the model kinase group, or showing high affinity to less than five kinases in the model kinase group) is predicted to test negative in either the in vitro or in vivo toxicity assay.

Candidate drugs that test positive in the assay of the invention (i.e., that are predicted to demonstrate cardiotoxicity in the in vitro or in vivo assays) would be flagged as a potential cardiotoxic compound and would be placed on hold, rejected or otherwise dropped from further development. In the case of high-throughput screening applications, such compounds can be flagged as potentially (for example, by the software managing the system in the case of an automated high-throughput system), thus enabling earlier decision making.

Thus, one can use the method of the invention to prioritize and select candidate compounds for pharmaceutical development based in part on the potential of the compound for cardiotoxicity. For example, if one has prepared a plurality of compounds (e.g., 50 or more), having similar activity against a selected target, and desires to prioritize or select a subset of said compounds for further development, one can test the entire group of compounds in the method of the invention and discard or reject all those compounds that exhibit positive signs of cardiotoxicity. This reduces the cost of pharmaceutical development, and the amount invested in any compound selected for development by identifying an important source of toxicity early on. Because the method of the invention is fast and easily automated, it enables the bulk screening of compounds that would otherwise not be possible or practical.

Environmental pollutants and the like can also be identified using the method of the invention, in which case such compounds are typically identified for further study into their toxic properties. In this application of the method of the invention, one can fractionate an environmental sample (for example, soil, water, or air, suspected of contamination) by known methods (for example chromatography), and subject said fractions to the method of the invention. Fractions that display signs of cardiotoxicity can then be further fractionated, and (using the method of the invention), the responsible toxic agents identified. Alternatively, one can perform the method of the invention using pure or purified compounds that are suspected of being environmental pollutants to determine their potential for cardiotoxicity. Because the method of the invention is fast and easily automated, it enables the bulk screening of samples that would otherwise not be possible or practical.

### EXAMPLES

The aim of the analysis was (1) to build a model using kinase inhibition profiles to predict cardiotoxicity and (2) to identify the kinases correlated with cardiotoxicity. The analysis was carried out in several steps: first, eighteen suitable internal and marketed small molecule kinase inhibitors (SMKIs0) were selected to form a training set with which to build the model; second, for each compound in the training set, a cardiotoxicity assessment (positive or negative) and single point inhibition profiles against 290 kinases were acquired; and third, a statistical analysis was performed to build a predictive model.

Inhibition profiles against 290 kinases and cardiotoxicity labels were acquired for each compound in the training set (N=18). Each compound was tested at 10 μM concentration and a compound was considered as inhibiting a given kinase if >80% inhibition of the kinase was measured. Of the eighteen compounds, eight were assessed as positive for cardiotoxicity and ten were assessed as negative as determined by information available from public literature and from internal data (Table 1).

TABLE 1

| Compounds | Cardiotoxicity | Target |
|---|---|---|
| Gleevec | Positive | Abl1/2, PDGFRa/B, Kit |
| Iressa | Negative | EGFR (ERBB1) |
| | | Raf-1/B-Raf, VEGFR2/3, PDGFRa/B, |
| Nexavar | Positive | Kit, FLT3 |
| Nilotinib | Positive | Abl1/2, PDGFRa/B, Kit |
| RO-5406 | Negative | n/a |
| RO-7710 | Negative | n/a |
| RO-9535 | Negative | EGFR, PDGFR, FGFRa, KDRa |
| RO-6145 | Negative | n/a |
| RO-7520 | Positive | multiple |
| RO-3604 | Negative | h-CASEIN KINASE 1 delta-E. coli-c |
| RO-5981 | Negative | multiple |
| RO-6226 | Negative | n/a |
| Vertex | Positive | multiple |
| Sprycel | Positive | Abl1/2, PDGFRa/B, Kit, SRC family |
| Sutent | Positive | VEGFR1/2/3, Kit/PDGFRa/B, RET, |
| | | CSF-1R, FLT3 |
| Tarceva | Negative | EGFR (ERBB1) |
| Tykerb | Negative | EGFR (ERBB1), ERBB2 |
| Zactima | Positive | VEGFR/EGFR |

Pre-processing was first performed across the set of all inhibition profiles to remove uninformative kinases. Kinases with no variance in percent inhibition across the set of 18 compounds were removed, as they were not informative in separating the cardiotoxicity labels.

Feature selection (FS) and pattern recognition (PR) were performed in several phases in order to build the model. For all analyses, cross validation was used to assess the model performance over several trials. Each trial randomly split the initial data into a training set and a test set; the training set was used to build the temporary model, and the test set was used to predict results and then verify performance. Each cross validation fold was stratified, that is, the proportion of positive to negative compounds was kept roughly equal across all folds. FS methods were then used to determine which kinases, or “features”, were likely to correlate most with cardiotoxicity labels. In each trial, the inhibition values against the features chosen were used as input for a PR method, when then predicted the positive or negative result.

In the first phase of this analysis, FS methods were divided into two groups: univariate filter methods appropriate for a large input data set (FS1), and multivariate methods that performed better with less data (FS2). Different combinations of FS1, FS2, and PR methods were tested over several trials using 25 four-fold stratified cross-validations. The combination of methods with the lowest mean error rate was chosen as the final method set for the model. This combination included Single Train Error for FS1, Random Forests for FS2, and Random Forests for classification for PR. This combination of machine learning methods correctly predicted cardiotoxicity labels with an accuracy of 87%, sensitivity of 85%, and specificity of 88%.

In the second phase, model kinases were chosen based on the how often they were selected as significant in the previous phase of model building when the optimal method combination was run. Over 25 trials of four-fold cross validation, each kinase could have been selected a maximum of 100 times, once per training set (4-fold cross validation, run 25 times). After building the model and assessing performance, the number of times a kinase was chosen as significant amongst the 100 trials was tallied. 86 of the original 290 kinases were chosen at least once. Those kinases that were chosen with a frequency greater than or equal to 50% (N=11) were chosen to be included in the final model. These kinases were: CSF1R, KIT, FYN, PDGFRB, FGR, LCK, EPHB1, FRK, ABL1, PDGFRA, and HCK.

Based on the statistical analysis of the original dataset of 18 compounds, it may be possible to expand the model kinases to include other kinases that were chosen as significant predictors in less than 50% of the test runs. This set of eleven additional kinases include: ABL2, LYN, ZAK, EPHB2, YES, MAP4K4, PKN1, BRAF, DDR2, MAP4K5, and STK24.

For each compound, the model consists of single point kinase inhibition profiles against the following eleven kinases: CSF1R, KIT, FYN, PDGFRB, FGR, LCK, EPHB1, FRK, ABL1, PDGFRA, and HCK. The eleven kinases in the model were chosen based on the statistical analysis of an initial training set of 18 compounds. Over multiple runs of testing, the kinase percent inhibition profiles against these eleven kinases were found to be significant in predicting cardiotoxicity at least 50% of the time. That is, compounds that were determined to be cardiotoxic tended to have high levels of inhibition against the eleven kinases. By using the kinase inhibition profile against these eleven kinases as input into a Random Forest classifier, an accurate prediction of cardiotoxicity can be determined.

Given the kinase inhibition profile of a compound against the eleven kinases, the model is used to predict whether that compound will be cardiotoxic. The information from the model results would be useful as a pre-screening for compounds, given the assessment difficulty and lack of mechanistic understanding of cardiotoxicity. Based on a preliminary training set of compounds with kinase inhibition profiles and known cardiotoxicity assessment, the model has performed with an accuracy of approximately 87%, with a sensitivity and specificity of 85% and 88%, respectively. With 50% accuracy being equivalent to random classification, this model has performed well and has proved its utility in predicting cardiotoxicity.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

What is claimed:

1. A method for predicting the cardiotoxicity of a compound, said method comprising:
a) providing a test compound;
b) determining the ability of the compound at a concentration of about 10 μM to inhibit the kinase activity in the model kinase group consisting of CSF1R, KIT, FYN, PDGFR beta, FGR, LCK, Ephrin Receptor B1, FRK, ABL1, PDGFR alpha, and HCK, wherein inhibition by over 80% of at least one of said kinases indicates a likelihood that said test compound will demonstrate cardiotoxicity.

2. The method of claim 1, wherein inhibition by over 80% of at least five of said kinases in the model kinase group indicates a likelihood that said compound will demonstrate cardiotoxicity.

3. The method of claim 1, wherein inhibition by over 80% of all eleven of said kinases in the model kinase group indicates a likelihood that said test compound will demonstrate cardiotoxicity.

4. The method of claim 1, wherein step b) further comprises determining the ability of the compound at 10 μM to inhibit the kinase activity in the potential model kinase group consisting of ABL2, LYN, ZAK, Ephrin Receptor B2, YES1, MAP4K4, PKN1, BRAF, DDR2, MAP4K5, and STK24, wherein inhibition by over 80% of at least one of said kinases in the potential model kinase group indicates a likelihood that said test compound will demonstrate cardiotoxicity.

5. The method of claim 4, wherein wherein inhibition by over 80% of at least five of said kinases in the model kinase group indicates a likelihood that said compound will demonstrate cardiotoxicity.

6. The method of claim 4, wherein inhibition by over 80% of all eleven of said kinases in the model kinase group indicates a likelihood that said test compound will demonstrate cardiotoxicity.

7. A method for screening compounds for potential cardiotoxicity, said method comprising:
a) providing a plurality of test compounds;
b) determining the ability of each compound at 10 μM concentration to inhibit the kinase activity in the model kinase group consisting of CSF1R, KIT, FYN, PDGFR beta, FGR, LCK, Ephrin Receptor B1, FRK, ABL1, PDGFR alpha, and HCK, wherein inhibition by over 80% of at least one of said kinases indicates a likelihood that said test compound will demonstrate cardiotoxicity;
c) rejecting compounds that demonstrate a likelihood of cardiotoxicity.

8. The method of claim 7 wherein inhibition by over 80% of at least five of said kinases in the model kinase group indicates a likelihood that said compound will demonstrate cardiotoxicity.

9. The method of claim 7 wherein inhibition by over 80% of all eleven of said kinases in the model kinase group indicates a likelihood that said test compound will demonstrate cardiotoxicity.

10. The method of claim 1 or claim 7, wherein the ability of the compound to inhibit the kinase activity is determined by measuring the binding affinity of the compound for said kinases.

11. A test substrate, comprising:
A solid support; and
Immobilized on said solid support, the kinases CSF1R, KIT, FYN, PDGFR beta, FGR, LCK, Ephrin Receptor B1, FRK, ABL1, PDGFR alpha, and HCK.

12. The test substrate of claim 9, further comprising:
Immobilized on said solid support, the kinases ABL2, LYN, ZAK, Ephrin Receptor B2, YES1, MAP4K4, PKN1, BRAF, DDR2, MAP4K5, and STK24.
